(19) United States
(12) Patent Application Publication
(10) Pub. No.: US 2007/0233088 A1
(43) Pub. Date: Oct. 4, 2007

(54) PEDICLE AND NON-PEDICLE BASED INTERSPINOUS AND LATERAL SPACERS

(76) Inventor: Elizabeth W. Edmond, Ann Arbor, MI (US)

Correspondence Address: GIFFORD, KRASS, SPRINKLE,ANDERSON & CITKOWSKI, P.C, PO BOX 7021, TROY, MI, 48007-7021, US

(21) Appl. No.: 11/668,029

(22) Filed: Jan. 29, 2007

Related U.S. Application Data
(60) Provisional application No. 60/762,586, filed on Jan. 27, 2006.

(51) Int. Cl. A61F 2/30

(52) U.S. Cl. 606/61

(57) ABSTRACT

Pedicle and non-pedicle based interspinous and lateral spacers have an upper surface configured for engagement with an inferior surface of a fifth lumbar vertebral body, and a lower surface configured for engagement with an outer surface of a sacrum. One configuration includes a component having two opposing upper arms and two opposing lower arms. The spacer component has two ends and a central section, each end of the spacer component being configured for attachment to a respective one of the two opposing upper arms, and the central section of the spacer has a height configured for placement between a spinous process of a fifth lumbar vertebral body and a superior surface of an uppermost spinous process of a sacrum. The ends of the spacer component may be attached to the upper arms using pedicle screws, or may use snap-and-lock or other connectors. The two lower arms may either engage directly with the outer surface of a sacrum on either side of a medial ridge, or may interconnect with a separate component also having two lower arms that engage with the outer surface of a sacrum on either side of a medial ridge. Such arms are preferably bent outwardly and include inward serrations to engage with the outer surface of a sacrum on either side of a medial ridge. Other configurations include a spacer component that engages with a sacral notch.

### CROSS-REFERENCE TO RELATED APPLICATIONS

This application claims priority from U.S. Provisional Patent Application Ser. No. 60/762,586, filed Jan. 27, 2006, the entire content of which is incorporated herein by reference.

### FIELD OF THE INVENTION

This invention relates generally to spinal stabilization devices and, in particular, to pedicle and non-pedicle based interspinous and lateral spacers.

### BACKGROUND OF THE INVENTION

Natural intervertebral discs serve multiple purposes. First, they preserve correct anatomical spacing between adjacent vertebral bodies, allowing branching nerve bundles to function normally, without pain. Discs also facilitate natural flexion, extension, and lateral bending in support of daily physical activities. Discs further serve as “shock absorbers” for spinal loading.

However, for many reasons, natural discs can loose functionality, often leading to back pain. These sources may include physical trauma, degenerative disc disease, and other maladies. Today there are many options to stabilize spinal segments which may exhibit a loss of functionality. One option is spinal fusion, wherein a complete or partial discectomy is performed, with one or more cages or other mechanical devices being inserted into the disc space. Another option gaining in popularity is the use of “artificial discs,” which typically include either a resilient central portion or mechanical elements that facilitate a certain degree of articulation.

Various types of intervertebral spacers are also available as valuable tools to promote spinal stabilization. Such devices may be used in conjunction with fusion, for example, to relieve pressure from the central vertebral column. Spacers may also be valuable in relieving spinal stenosis and other conditions resulting in back pain.

### SUMMARY OF THE INVENTION

This invention is directed to pedicle and non-pedicle based interspinous and lateral spacers. Each embodiment includes some form of spacer component having an upper surface configured for engagement with an inferior surface of a fifth lumbar vertebral body, and a lower surface configured for engagement with an outer surface of a sacrum.

A first preferred embodiment includes a component having two opposing upper arms and two opposing lower arms. The spacer component has two ends and a central section, each end of the spacer component being configured for attachment to a respective one of the two opposing upper arms, and the central section of the spacer has a height configured for placement between a spinous process of a fifth lumbar vertebral body and a superior surface of an uppermost spinous process of a sacrum. The ends of the spacer component may be attached to the upper arms using pedicle screws, or may use snap-and-lock or other connectors.

The two lower arms may either engage directly with the outer surface of a sacrum on either side of a medial ridge, or may interconnect with a separate component also having two lower arms that engage with the outer surface of a sacrum on either side of a medial ridge. Such arms are preferably bent outwardly and include inward serrations to engage with the outer surface of a sacrum on either side of a medial ridge.

A different embodiment includes two spacer components, one on each end of a horizontal bar, each spacer component having a lower, concave end configured for engagement with a sacral notch. The horizontal bar preferably further includes a pair of downwardly extending elements, each being bent outwardly to engage with the outer surface of a sacrum on either side of a medial ridge.

In a further embodiment the spacer component includes an upper concave surface configured to engage with the inferior surface of a transverse process and a lower concave surface configured to engage with a sacral notch. A link member may be provided to connect the spacer component to a pedicle using a pedicle screw, for example.

### BRIEF DESCRIPTION OF THE DRAWINGS

FIG. 1A is a posterior view of an interspinous spacer according to the invention;

FIG. 1B is a drawing of a preferred interspinous insert;

FIG. 1C is a side view of the insert of FIG. 1B;

FIG. 1D is a side view of an alternative insert utilizing snap/lock interconnects;

FIG. 2A is a drawing of an alternative spacer that facilitates stacking;

FIG. 2B is a lateral view of the arrangement of FIG. 2A, showing sacral engagement features;

FIG. 3A is the posterior view of an alternative interspinous spacer according to the invention;

FIG. 3B is a side view showing the way in which a lower sacral portion is bent for enhanced anatomical engagement;

FIG. 3C is a posterior view drawing showing how the arms of FIG. 3A may be hinged through interlocking;

FIG. 4A is a drawing showing a sacral notch sling according to the invention;

FIG. 4B is side view of the device of FIG. 4A;

FIG. 4C is a detailed view showing the way in which the device of FIG. 4A cooperates with a sacral notch;

FIG. 5A is a posterior view of a pedicle-based lateral spacer according to the invention;

FIG. 5B is a detailed drawing showing the way in which the spacer of FIG. 5A cooperates with a sacral notch and the inferior surface of a transverse process;

FIG. 6A is a posterior view of lateral spacers that do not require fasteners such as pedicle screws; and

FIG. 6B is a detailed drawing showing the way in which the lateral spacers of FIG. 6A cooperate with a sacral notch and the inferior surface of a transverse process.

### DETAILED DESCRIPTION OF THE PREFERRED EMBODIMENTS

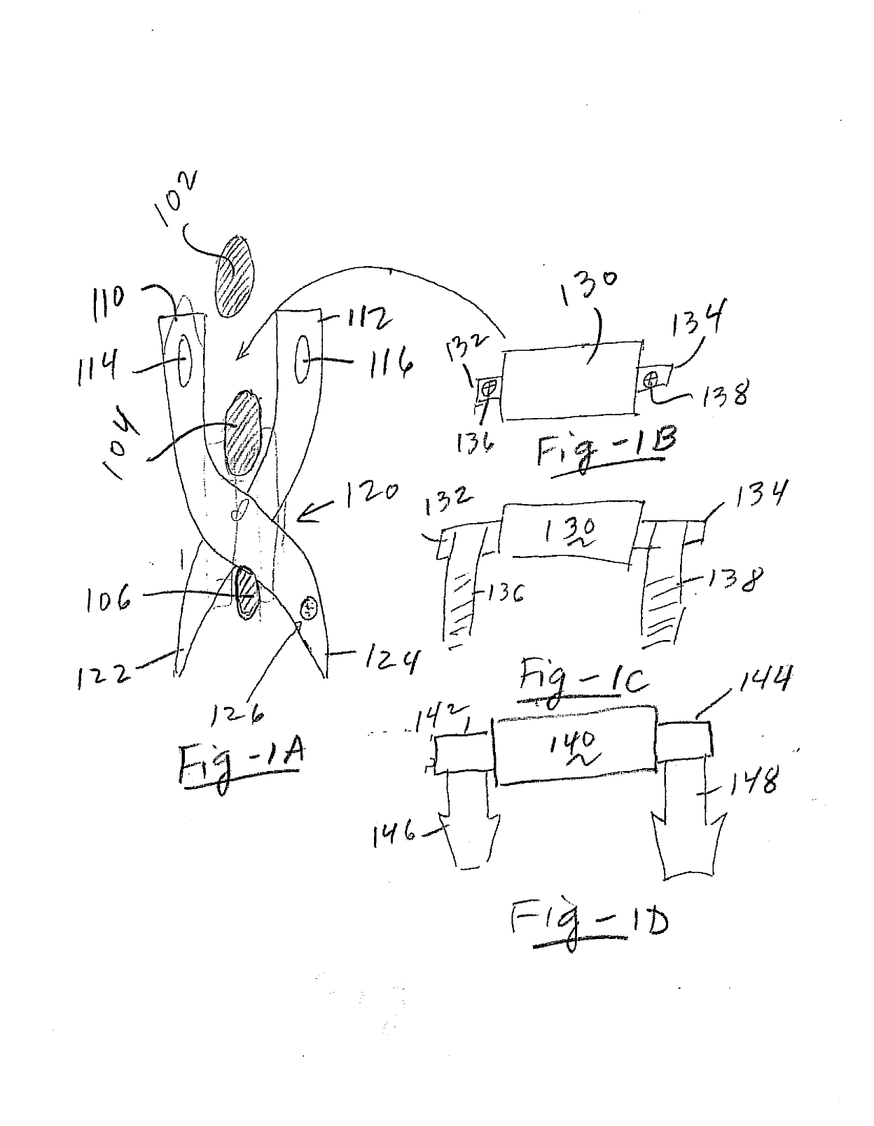

Turning now to the drawings, FIG. 1A is a posterior view of an interspinous spacer system according to the invention. The device includes first and second upper arms 110, 112, which intersect a region 120 between spinous process 104 of L5 and 106, the upper spinous process of the sacrum, sometimes referred to as the upper spinous tubercle on the medial sacral crest. The device further includes first and second lower arms 122, 124 which continue down past 106 and engage with the surface of the sacrum on either side of the medial sacral crest.

The arms may extend from a unitary structure, or two crossing pieces may be used, in which case they may be permanently joined or hinged to accommodate varying recipient physiology. One or more removable insertion pegs 126 may be located as shown, or with respect to other portions of the device.

The upper arms 110, 112 in this embodiment include holes or slots 114, 116, which receive a spacer between spinous process 102 of L4 and spinous process 104. FIG. 1B shows a preferred spacer, including a central portion 130 physically configured to achieve the desired level of separation or distraction, with outward arms 132, 134. Pedicle screws 136, 138 are provided through the arms 132, 134, and the screws 136, 138 continued through apertures 114, 116, into pedicles (not shown). This creates a highly stable configuration in the lower lumbar and sacral region.

FIG. 1C is a side-view drawing of the spacer of FIG. 1B, showing pedicle screws 136, 138 with their threaded portions. FIG. 1D is a side-view drawing of an alternative spacer according to the invention, having a central distraction portion 140 and outward arms 142, 144 to which they are connected snap-in ends 146, 148 which cooperate with apertures 114, 116 without the need for pedicle screws.

In these embodiments, all of the components can be made out of any appropriate biocompatible materials, such as metals like chrome-cobalt, titanium, or the like, ceramics, or compressible/resilient materials where appropriate, assuming acceptable longevity. To install the spacer, it is assumed that the supraspinous ligament is sacrificed between spinous processes 102, 104. However, the supraspinous ligament may be preserved in the region of 120. Arms 122, 124, preferably include serrations to engage with outer sacral surfaces as discussed in further detail below.

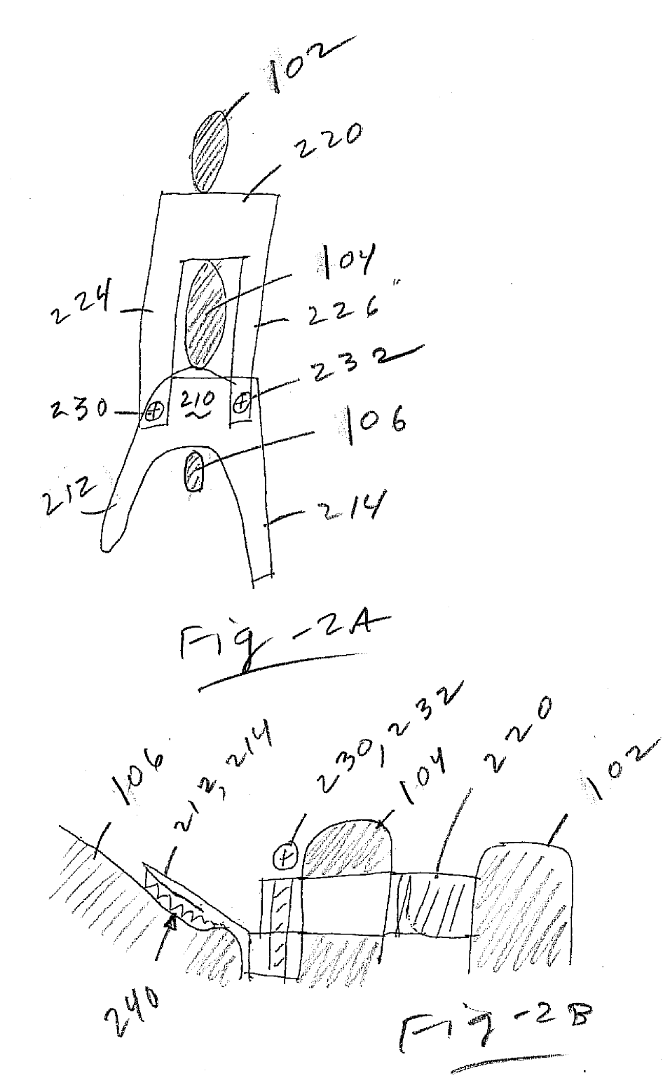

FIG. 2A is a posterior view of an alternative embodiment of the invention, showing the way in which separate components may be stacked to achieve a result similar to that introduced with respect to FIG. 1. In this embodiment, an upper portion 220, having lower arms 224, 226, separates spinous processes 102, 104. At the lower ends of the arms 224, 226, are holes to receive pedicle screws 230, 232 which penetrate through a separate unit 210 disposed between spinous process 104 and spinous process 106 present on the sacrum. Component 210, in turn, includes lower arms 212, 214, preferably with serrations 240, shown in FIG. 2B, which engage with the sacrum 106. FIG. 2B is a side-view drawing and partial cross-section better illustrating anterior-posterior (A-P) positioning of the components depicted in FIG. 2A.

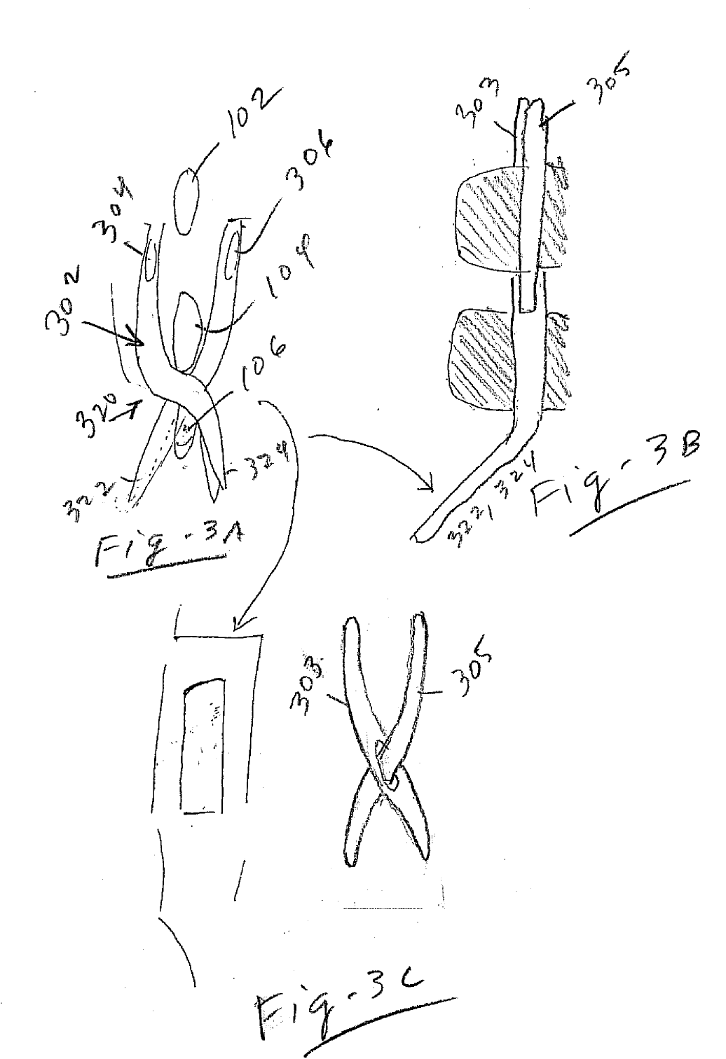

FIG. 3A is a posterior view drawing of a further alternative embodiment of the invention, showing how a criss-cross or scissors-like device 302, similar to that introduced with respect to FIG. 1, may be used with bent lower portions 322, 324 for conformity with the sacral surface, as better seen in FIG. 3B. Such a structure also facilitates interlocking separate arms 303, 305, as shown in FIG. 3C. A spacer of the types depicted in FIG. 1 is used between the spinous processes 102, 104, engaging with apertures or slots 304, 306. Again, in the region 320, the arms may join in unitary fashion, or a hinged configuration may be used.

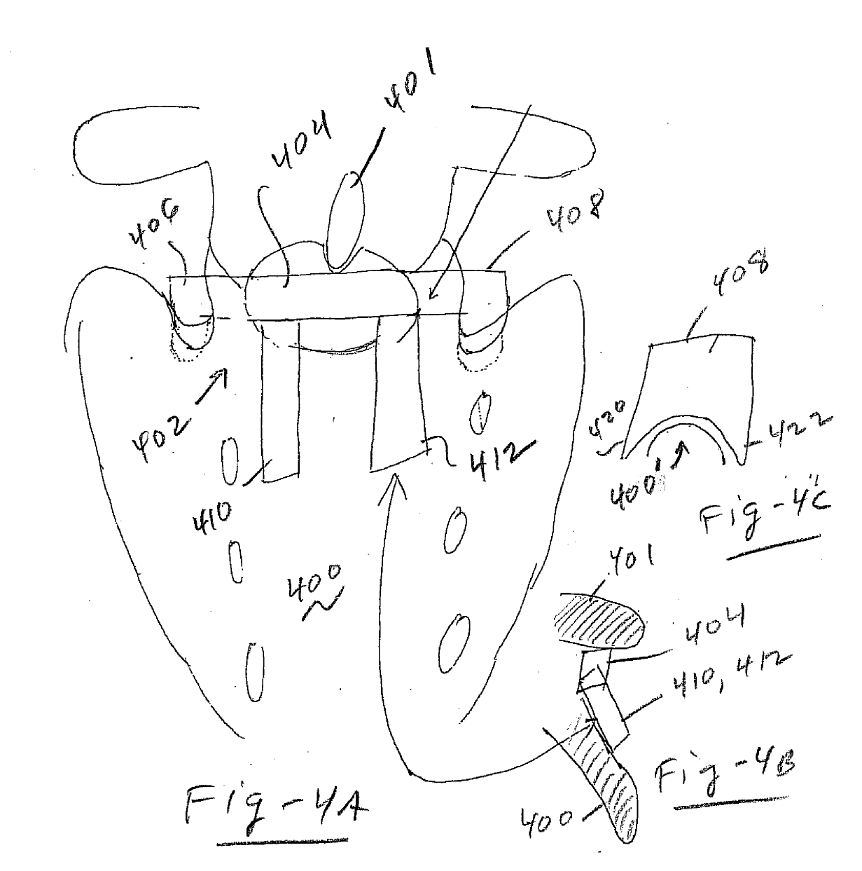

FIG. 4A is a posterior view drawing of yet a further embodiment of the invention in the form of a sacral notch sling. The spinous process of L5 is shown at 401. The device, 402, includes a central bar portion 404, having ends 406, 408, which engage with the sacral notch 400′ on either side, as best shown in FIG. 4C. The sacral notch is defined as the concave groove between the sacral ala and sacral horns or superior articular processes on either side of the sacrum. The outer surface of the groove is generally smooth and convex.

From the lateral view of FIG. 4C, it can be seen portions 420, 422 have concave surfaces oriented on either side of the outer surface of the sacral notch 400′. The device 402 further includes lower arms 410, 412, which, as in other embodiments, may be bendable to suit different patients and which may include serrations (not shown) to better engage with the other surface of the sacrum on either side of the medial crest.

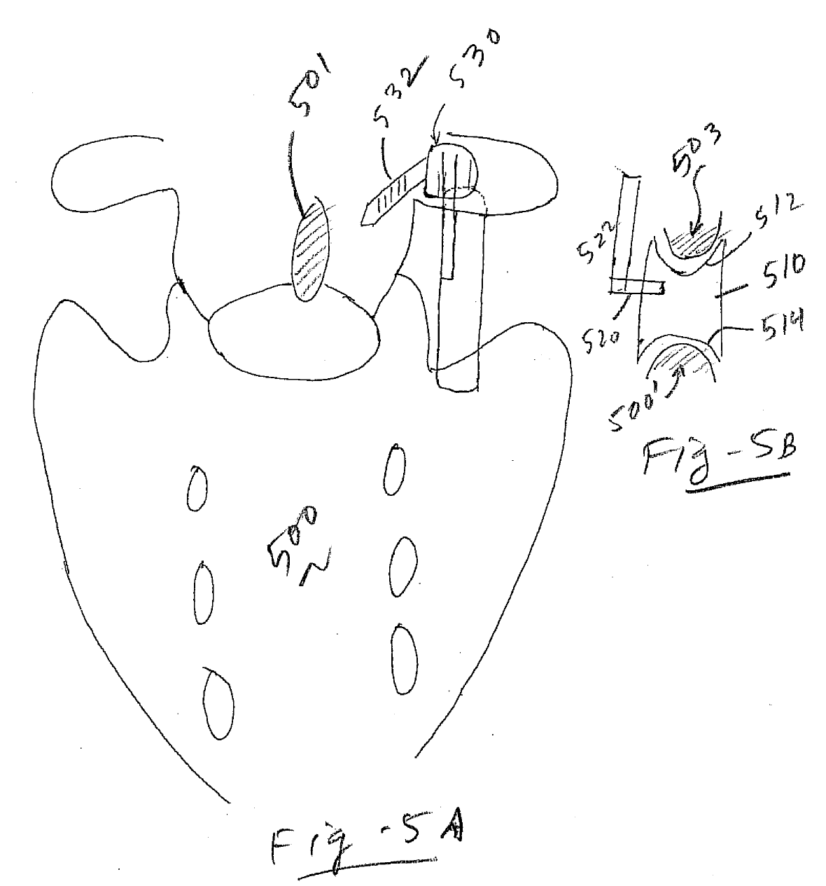

FIG. 5A is a posterior view drawing of yet a different embodiment of the invention, in the form of a lateral spacer 510, including a pedicle-based fixation arrangement. The spinous process for L5 is shown at 501, and the sacrum is indicated generally at 500. The spacer, 510, best seen in FIG. 5B, includes a superior portion 512 having a surface that engages with the inferior surface of the transverse process 503, and a lower surface 514 engages with the superior surface of the sacral notch 500′.

One or more elements are interconnected to the spacer 510, such as horizontal arm 520 which in turn connects to a vertical arm 522 which, in turn, attaches to a receptacle 530 for a pedicle screw 532. The various links 520, 522, 530, and so forth, may be adjusted laterally or from anterior to posterior, to ensure that the spacer 510 is correctly positioned, and that the pedicle screw 532 is properly received. Although a single spacer is shown in FIG. 5A, it will be appreciated that the spacer on the other side, or on both sides, may be accommodated depending upon the case.

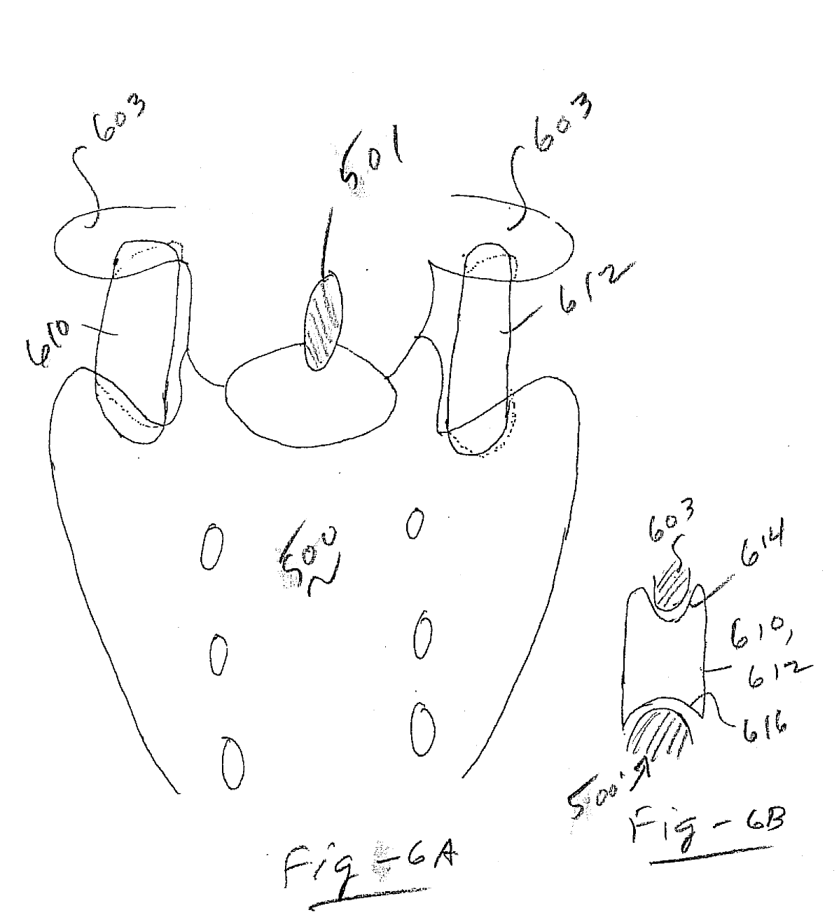

FIG. 6A is a posterior view drawing of yet a further, different embodiment of the invention, in the form of more simplified spacer 610, 612, which do not require the use of fasteners such as pedicle screws. Again, the spinous process for L5 is shown at 501, and the sacrum is shown at 500. FIG. 6B is a lateral view, showing how the spacer 610, 612, each have a superior surface 614 that interfaces to a transverse process 603, and a lower portion 616 which conforms to sacral notch 600, 500′. Although only a single one of the spacers, 610, 612, may be used, they are generally used in pairs due to the lack of fasteners.

I claim:

1. A spinal stabilization system, comprising:
a spacer component, including:
an upper surface configured for engagement with an inferior surface of a fifth lumbar vertebral body; and
a lower surface configured for engagement with an outer surface of a sacrum.

2. The spinal stabilization system of claim 1, further comprising:
a component having two opposing upper arms and two opposing lower arms; and wherein:
the spacer component has two ends and a central section, each end of the spacer component being configured for attachment to a respective one of the two opposing upper arms; and
the central section of the spacer has a height configured for placement between a spinous process of a fifth lumbar vertebral body and a superior surface of an uppermost spinous process of a sacrum.

3. The spinal stabilization system of claim 2, wherein the ends of the spacer component are attached to the upper arms using pedicle screws.

4. The spinal stabilization system of claim 2, wherein the ends of the spacer component are attached to the upper arms using snap and lock connectors.

5. The spinal stabilization system of claim 1, wherein:
the spacer component includes two lower arms that engage with a separate component also having two lower arms that engage with the outer surface of a sacrum on either side of a medial ridge.

6. The spinal stabilization system of claim 5, wherein the lower arms of the separate component are bent outwardly and include inward serrations to engage with the outer surface of a sacrum on either side of a medial ridge.

7. The spinal stabilization system of claim 1, including:
two spacer components, one on each end of a horizontal bar; and
wherein each spacer component includes a lower, concave end configured for engagement with a sacral notch.

8. The spinal stabilization system of claim 7, further including a pair of downwardly extending elements, each being bent outwardly to engage with the outer surface of a sacrum on either side of a medial ridge.

9. The spinal stabilization system of claim 1, wherein the spacer component includes an upper concave surface configured to engage with the inferior surface of a transverse process and a lower concave surface configured to engage with a sacral notch.

10. The spinal stabilization system of claim 1, wherein:
the spacer component includes an upper concave surface configured to engage with the inferior surface of a transverse process and a lower concave surface configured to engage with a sacral notch; and
a link member connecting the spacer component to a pedicle using a pedicle screw.
